(19) United States
(12) Patent Application Publication
(10) Pub. No.: US 2006/0024660 A1
(43) Pub. Date: Feb. 2, 2006

(54) Method for relative quantification of proteins

(76) Inventors: Patrick Jouin, Saint Clement De Riviere (FR); Nathalie Galeotti, Fabregues (FR)

Correspondence Address: FISH & RICHARDSON PC, P.O. BOX 1022, MINNEAPOLIS, MN, 55440-1022, US

(21) Appl. No.: 10/900,262

(22) Filed: Jul. 27, 2004

(51) Int. Cl. C12Q 1/00; G01N 33/00

(52) U.S. Cl. 435/4; 436/86

(57) ABSTRACT

The invention relates to a method for comparative analysis of two or more samples to determine ratios of the amounts of proteins of the samples, which method comprises labeling lysine residues of the proteins, separating the labeled proteins by 2D gel electrophoresis, before cleavage and mass spectrometry analysis.

This invention relates generally to labeling methods for quantitative proteomics, and more specifically to in vitro pre-digestion Lys specific tagging methods for determining the relative quantities of peptides and proteins using mass spectrometry.

### BACKGROUND OF THE INVENTION

The proteome was defined as the complete set of proteins expressed in a given cell at a given time. However the term “proteomics” is often used as a large-scale approach to study proteins. Often one of the important aspects in proteomic studies is the need to measure the relative amount of proteins. This is essential for studying the effect on an agent on a biological system or for the comparison of two different biological states.

The most traditional approach for quantifying proteins in a proteomic experiment is based on statistical analysis to measure the ratios of the protein spot intensities in two independent set of gels that are colored by coomassie or silver stain, or revealed by fluorescent dyes. Recent developments involve analysis on a single gel containing mixed sampled differentially labelled with three independant fluorophores (DIGE).

Alternative approaches rely on mass spectrometry (MS) to measure relative abundances of proteins. Quantification by MS requires production of differentialy labelled peptides from two sets of proteins whose ratio is obtained by measuring the relative intensities of the MS signals of the mixed labelled peptides. Different strategies have been disclosed (Flory, Griffin et al. Trends Biotechnol. 2002 Dec., 20(12 suppl):S23-9; Hamdan and Righetti 2002, Mass Spectrom Rev. 21(4):287-302; Moritz and Meyer 2003 Proteomics 3(11):2208-20; Sechi S. Contrib Nephrol. 2004, 141:59-78).

Post-extraction chemical modifications have been introduced to differently label proteins on a global level after they have been extracted from the cell with distinctive residue selective isotopic tags. For post-extraction isotopic labeling only selective alkylation of cystein residues with either a heavy or light reagent have been described. The best known methodology utilizes a reagent referred to as isotope-coded affinity tags (ICAT Gygi et al., R. Nat. Biotechnol. 17, 994-999 (1999)). ICAT-labelled peptides are further isolated by affinity chromatography and then analyzed by on-line HPLC coupled to tandem MS. Using unlabeled and deuterium-labeled acrylamide, cystein alkylation prior to gel electrophoresis isolation of proteins was also used as a tool for identification and quantitation on MALDI MS (Sechi, S., Rapid Commun. Mass Spectrom. 16, 1416-1424 (2002)).

### SUMMARY OF THE INVENTION

The present invention provides a simple method for quantification of proteins or peptides in complex mixtures.

The invention relates to a method for comparative analysis of two samples to determine ratios of the amounts of proteins of the samples, which method comprises labeling lysine residues of two protein pools with two different but closely chemically related reagents, mixing of the two differentialy labelled pools of proteins, separating the labeled proteins, preferably by 2D gel electrophoresis, before cleavage and mass spectrometry analysis.

The method of the invention preferably comprises the steps of:
(i) labeling ε-amino groups of lysine residues of the proteins of a first sample, with a first amidination agent;
   labeling ε-amino groups of lysine residues of the proteins of a second sample, with a second amidination agent;
wherein the first amidination agent and the second amidination agent are selected to give a desired mass shift by mass spectrometry;
(ii) mixing pools of the first and second samples;
(iii) separating the labeled proteins, preferably by 2D gel electrophoresis;
(iv) cleaving the separated proteins;
(v) subjecting the cleaved proteins to mass spectrometry analysis.

Preferably the first amidination agent differs from the second amidination agent by at least a methylene group (14 u). More preferably the first amidination agent is S-methyl thioacetamidate and the second amidination agent is S-methyl thiopropinimidate. These labels are introduced as variable modifications within the software used for protein identification, preferably Mascot® (MATRIX SCIENCE).

The proteolysis step iv) is preferably a trypsin digestion.

The mass spectrometer is advantageously a MALDI-TOF but is compatible with any other kind of source versus detector configuration (matrix assisted laser desorption ionization (MALDI), electron spray ionization/time-of-flight mass (ESI/TOF), quadrapole (O), ion trap (Trap), Fourier transform mass spectometer (FT).

The method of the present invention can be applied to determine the relative quantities of one or more proteins in two or more protein samples which are analyzed by pairs.

In a preferred embodiment, the samples are plant extracts.

Additional objects, advantages, and features of the present invention will become apparent from the following description taken in conjunction with the accompanying drawings.

### BRIEF DESCRIPTION OF THE DRAWINGS

FIG. 1 is a schematic drawing illustrating the acetamidination and propionamidination of Lys ε-amino groups.

FIG. 2 is a graph showing the ESI-QTOF spectra of labeled myoglobin before trypsin proteolysis.

FIG. 3A is a MALDI-TOF mass spectra comparing acetamidinated to propionamidinated ovalbumin trypic digests (ratio 7:3 and 3:7).

FIG. 3B is a graph showing the experimental intensity ratio vs. calculated concentration ratio of acetamidinated to propionamidinated ovalbumin trypic digests.

FIG. 4 is a score diagram from a Mascot® search showing identification of labeled proteins from Arabidopsis thaliana.

### DETAILED DESCRIPTION OF THE INVENTION

The present invention provides a method for protein identification and quantification of proteins or peptides in complex mixtures. The method is particularly applicable in comparative proteomics where the difference in concentration of peptides/proteins common to at least two different samples is detected.

The inventors have more particularly showed that a mass spectrometry analysis of proteins that have been labeled on lysine residues before trypsin digestion enables quantitative comparisons of protein mixtures.

Labeling lysines before digestion leads to the modification of the trypsic digestion profiles, which renders the spectrometry analysis easy to read, insofar as less peptides are produced upon subsequent digestion.

The decrease of complexity of the peptide mixtures being analyzed is therefore one of the main advantages of the invention.

The samples to be analyzed may be of any source. It may be a biological sample from an animal, or a plant extract. It may be a crude sample that this is likely to contain proteins or it may be a protein preparation.

It should be understood that the term “protein”, as used herein, refers to a polymer of amino acids and does not connote a specific length of a polymer of amino acids. Thus, for example, the terms oligopeptide, polypeptide, and enzyme are included within the definition of “protein”, whether produced using recombinant techniques, chemical or enzymatic synthesis, or naturally occurring. This term also includes polypeptides that have been modified or derivatized, such as by glycosylation, acetylation, phosphorylation, and the like. The term “peptide” as used herein, usually refers to shorter polymers of amino acids. Peptides may be naturally formed within a sample of the present invention or may be formed by cleavage of larger proteins by methods known to the skilled artisan. It will be appreciated that the method of the present invention can be applied to peptides, proteins or mixtures thereof. Therefore, reference to protein or peptide mixtures is not intended to be limiting.

The method of the invention is designed for use in complex samples containing a number of different proteins. The sample may contain a single protein or it may include all the proteins found in a total cell lysate. A sample can therefore include total cellular protein or some fraction thereof. For example, a sample can be obtained from a particular cellular compartment or organelle, using methods such as centrifugal fractionation. The sample can be derived from any type of cell, organism, tissue, organ, or bodily fluid, without limitation.

The samples to be compared may be a control sample and a experimental sample. In particular the method of the invention allows for analysis of cellular protein expression in two different samples. For instance one sample may be treated to induce expression, or different cellular growth conditions may be used to manipulate protein expression.

The method of the present invention allows for analysis of protein expression without having to actually isolate the protein of interest.

### Labeling of the Lysine Residues

The ε-amino groups of lysine residues of the proteins of the samples are differentially labeled with amidine moieties. The samples to be compared are labeled with a labeling agent that is selected to be different from one sample to another. The reagents are selected to give a desired mass shift. The labeling agents advantageously are amidination agents wherein the amidine moieties differ by a methylene group. The amidination agent may be a S-methyl thioacylimidate synthetized by methylation of the corresponding primary or secondary thio-amide, wherein the acyl group may be selected from the group consisting of acetyl, propionyl, butyryl, valeryl, hexynyl, isobutyryl and isovaleryl. More generally, the amidination agent may be a S-alkyl thioacylimidate, wherein the acyl group is as defined above, and wherein the alkyl group preferably comprises from 1 to 4 carbon atoms and is e.g. selected from the group consisting of methyl, ethyl, isopropyl, or t-butyl, said alkyl group being advantageously a methyl group. Thio-amide compounds with ramified or cyclic chain from one to five carbon atoms are further encompassed.

An amidination agent is preferably S-methyl thioacetamidate or S-methyl thiopropionimidate.

At least a determined portion of each sample, e.g. control and experimental samples, is then mixed together to yield a combined sample.

### Separation of the Labeled Proteins

The proteins so labeled in the combined sample are then separated, e.g. by preferentially 2D or 1D gel electrophoresis, or any other separation method.

Any staining procedure can be applied to visualize the proteins.

The great advantage of 2D electrophoresis is that it can separate several thousand proteins and provide a very good two dimensional display of a large number of proteins.

The electrophoresis is preferably carried out in a polyacrylamide gel.

In a preferred embodiment, the polyacrylamide gel electrophoresis is carried out in two dimensions with the first dimension is by isoelectric focusing and the second dimension is by SDS (sodium dodecyl sulphate) polyacrylamide gel electrophoresis.

Typical electrophoresis buffers (e.g. Hochstrasser et al. Anal. Biochem. 173:424 (1988) and O'Farrel, J. Biol. Chem. 250:4007 (1975)) contain components (e.g. tris(hydroxymethyl)aminomethane buffers and sodium dodecyl sulphate), that suppress the ionization of proteins in the mass spectrometer. These components may be replaced with other more volatile components (e.g. morpholinoalkylsulfonate buffers and ephemeral surfactants) that do not suppress ionization in the MS. In another embodiment, the samples are diluted with ammonium bicarbonate or ammonium acetate buffer to provide a volatile proton source for the mass spectrometer (Wilm, M et al. Anal. Chem. 68:1-8 (1996)).

### Cleavage

To aid in the mass spectral analysis, proteins in the samples to be compared are cleaved. Cleavage in solution can be achieved using any desired method, such as by using chemical, enzymatic, or physical means. Cleavage refers to scission of a chemical bond within peptides or -proteins in solution to produce peptide or protein “cleavage fragments”. Cleavage and the formation of peptide cleavage fragments in solution are to be differentiated from similar fragmentation processes in the gas phase within a mass spectrometer.

Cleavage of proteins can be achieved by chemical, enzymatic or physical means, including, for example, sonication or shearing. Preferably, the cleavage is a proteolysis. A protease enzyme is then used, such as, but not limited to, trypsin, chymotrypsin, papain, Arg-C, Glu-C, endo Lys-C, proteinase K, carboxypeptidase, calpain, subtilisin, staph V8 protease and pepsin. More preferably, a trypsin digestion is performed. Alternatively, chemical agents such as cyanogen bromide can be used to effect cleavage. The proteolytic agent can be immobilized in or on a support, or can be free in solution. Proteolytic enzymes and agents are well known in the art and the skilled artisan may chose the enzyme and agent based on the sample and the number of peptides desired.

### Mass Spectrometry

Mass spectrometric analysis is used to determine peak intensities and quantitate ratios in the combined sample, or determine whether there has been a change in the concentration of a protein between two samples. Preferably, changes in peptide concentration between the control and experimental samples are determined by their ratios using MALDI-mass spectrometry because MALDI-MS allows the analysis of more complex peptide mixtures, but ESI-MS may also be used.

The MALDI-MS is discussed in greater detail below. The matrix-assisted laser-desorption time-of-flight mass spectrometry (MALDI-TOF MS) is used to determine accurately the molecular weights (less than 100 parts-per-million (ppm)) of modified and unmodified peptides.

For a successful MALDI mass measurement, analytes are incorporated into UV-absorbing matrix crystals.

In the case of 2-D PAGE samples, a gel plug containing the stained protein is generally excised and transferred to a microvial for subsequent processing. The typical steps that follow can be summarized as: 1) destaining, 2) proteolytic digestion, 3) extraction of the resulting peptides from the gel plug, 4) desalting and concentration, and 5) dispensing a peptide/ionizing matrix mixture onto the surface of the MALDI target.

Following the MALDI-TOF MS analysis the identified masses excluding those ignored can be collected and used to search a listing containing sequence information produced by calculating in silico fragments which would be obtained by proteolysis of proteins of interest with an appropriate endopeptidase or chemical. The data collected using MALDI-TOF (MS) is represented as a list of parent ion masses. Masses due to the presence of the capture agent can be ignored and analysis focused on masses arising from the target peptide fragments. Intensities of each mass (m/z) feature in the mass spectrum are measured by methods known to those skilled in the art. Where more than one target peptide fragment is available for each protein of interest the intensities of masses in the mass spectra of such fragments can be normalized across such a plurality of signals resulting in greater accuracy and reliability.

Only as necessary, when further confirmatory data is required, further analysis of the sample can be performed using any standard method of tandem mass spectrometry (MS/MS) and in particular using MALDI-TOF/TOF (Ultraflex, Bruker or Applied Biosystems, Framingham, Mass.) or MALDI II Q-TOF (Micromass) or Q-STAR (Sciex) all of which are systems which continue MALDI MS with tandem mass spectrometry. This generates a fragmentation spectrum which is used to generate sequence information.

The foregoing and other aspects of the invention may be better understood in connection with the following examples, which are presented for purposes of illustration and not by way of limitation.

### EXAMPLES

### Example 1

### Quantitative Analysis of a Mixture of Ovalbumin and Myoglobulin

1.1 Materials and Methods

Validation experiments were performed on mixtures of commercial proteins (ovalbumin and myoglobin). Each protein was independently treated with S-methyl thioacetimidate (reagent 1) and S-methyl thiopropionimidate (reagent 2), synthetized as described in Beardsley R. and Reilly J. (2003), J. Proteome Res. 2, 15-21.

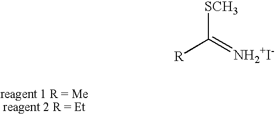

For the protein derivatization:

Labeling of ovalbumin— 30 μg of ovalbumin (Chicken egg) were dissolved in 10 μl of 6M urea. Protein was incubated at room temperature during 1 h 30 in the presence of 10 μl of either S-Methyl thioacetimidate and S-Methyl thiopropionomidate reagent (1 mg in 50 μl of 150 mM NaHCO₃). 2 μl of ice cold TCA (trichloro acetic acid) were added to the mixture and left 2 h. at 4° C. 38 000 g/30 min centrifugation was then performed. Trichloroacetic acid precipitate was then washed three times with diethyl ether.

The amidine labels using reagent 1 and reagent 2 differed by 14 da mass units. Samples of the labeled proteins were then mixed in various ratios. More particularly, six different samples were prepared by mixing methyl and ethyl-coded ovalbumin and myoglobin in ratios of 7:3, 5:5 and 3:7. The proteins were then separed on a 2D acrylamide gel electrophoresis.

2D acrylamide gel electrophoresis is well known in the art. Typical conditions are described in: Becamel C, Galeotti N, Poncet J, Jouin P, Dumuis A, Bockaert J, Marin P., Biol Proced Online. 2002 Dec. 9; 4:94-104.

Proteins of interest were excised and digested in gel using trypsin (sequencing grade, Promega, Madison, Wis.). Digest products were completely dehydrated in a vacuum centrifuge and resuspended in 10 ml formic acid (2% v/v), desalted using Zip Tips C18 (Millipore, Bedford, Mass.), eluted with 10 ml acetonitrile:trifluoroacetic acid, (80:0.1%) and concentrated to 2 ml. Aliquots of analyte solutions were mixed with the same volume of α-cyano-4-hydroxy-trans-cinnamic acid (10 mg/ml in acetonitrile:trifluoroacetic acid, 50:0.1%) and loaded on the target of a Bruker Ultraflex MALDI-TOF/TOF mass spectrometer (Bruker-Franzen Analytik, Bremen, Germany) using the Dry-droplet procedure (Matrix and sample mixed and dried on probe. Good for peptides, water-soluble samples. Karas M. and Hillenkamp F., Anal. Chem. 60, 2299 (1988); Cohen SL, Chait BT. Anal Chem. 68(1):31-7 (1996)). Spectra were analyzed using the XTOF software (Bruker-Franzen Analytik) and autoproteolysis products of trypsin (m/z 842.51, 1045.56, 2211.10) were used as internal calibrates. Identification of proteins are performed using MASCOT on a local server. Specific parameters are fixed for interrogation: acetimidoyl and propionimidoyl as fixed modifications; oxydation (M) as variable modification; Arg-C as enzyme (selected in the software for protein identification, insofar as Lys derivatization leads to ArgC line trypsin digestion); 0 missing cleavage; 50 ppm as peptide tolerance.

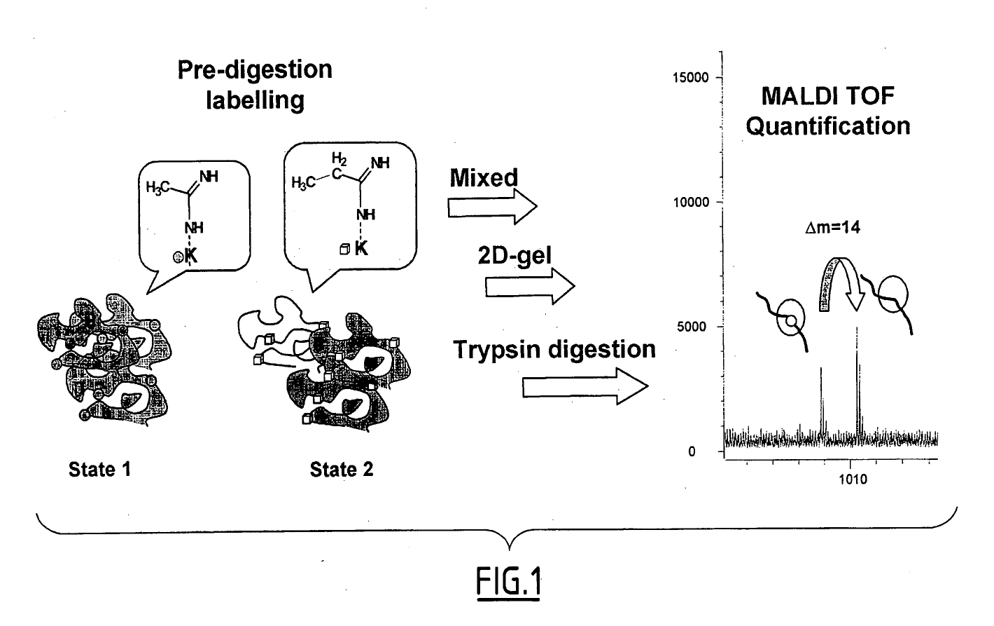

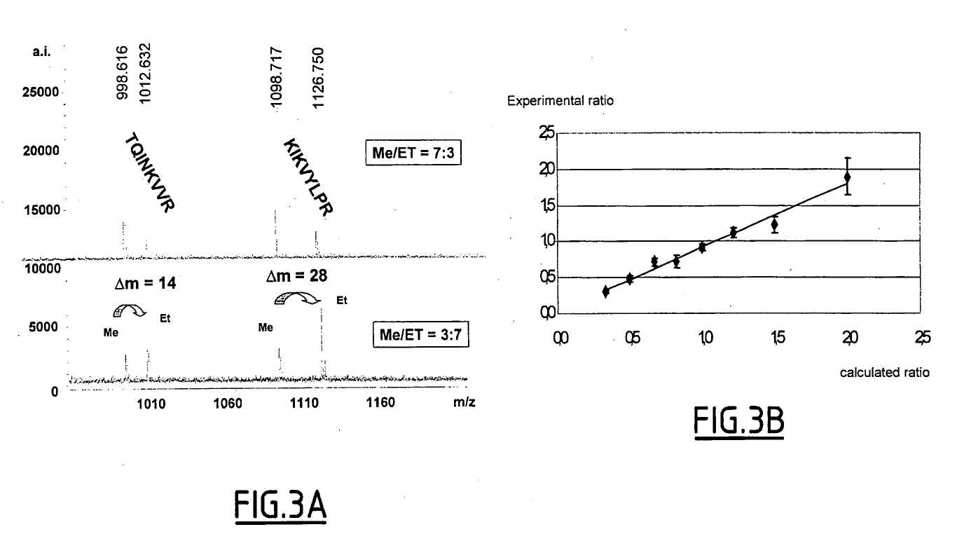

The labeled peptides appear as duplets with a mass difference being a multiple of 14 Da determined by the number of lysine residues in the peptide (see FIG. 1 and FIG. 3A). These mass differences are superior to those obtained in most differential labeling using stable isotopes.

1.2 Results

1.2.1 Derivatization Yield

Using ESI-QTOF mass analysis, the inventors showed that derivatization of lysine residues was quantitative. The experiment was performed on a sample of myoglobin labeled with reagent 1. Nanoelectrospray mass spectrometry was performed on a quadrupole time-of-flight (Q-TOF) mass spectrometer (QSTAR; Sciex, Toronto, Canada) equipped with a nanospray source (Protana Inc., Odense, Denmark).

Analysis of the signal that corresponds to a 21 charged ion shows the presence of a peak that is representative of acetamidate on 19 Lys residues and the amino-terminus.

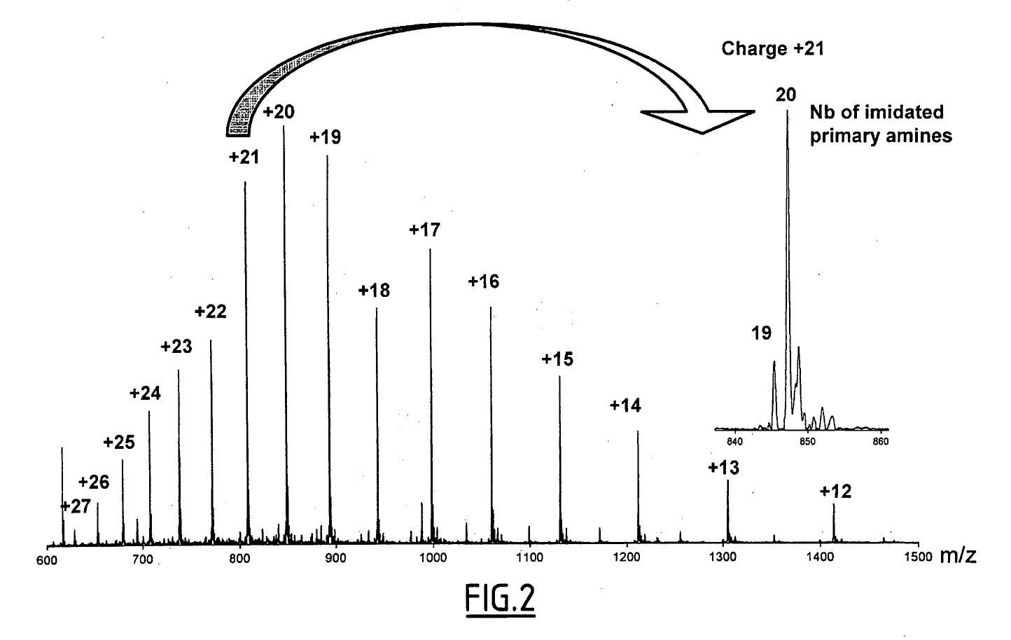

FIG. 2 shows ESI-QTOF spectra of the labeled myoglobin.

1.2.2 Analysis of 2D Gels

Labeled proteins were resolved by 2D electrophoresis giving a slight pl shift compared to unlabeled ones. This effect is only marked for low molecular weight proteins (<20 kd) with a high Lys content. In the given example myoglobin 17 kd; 19 Lys and ovalbumin 43 kd; 20 Lys.

This slight shift should not affect the correlation between the maps of labeled proteins and the maps of unlabeled proteins.

Moreover correlation is not needed for identifying the proteins, and quantification is not affected.

Finally, no pl or mass shift is observed between both methyl and ethyl-coded protein.

1.2.3. Trypsin Digestion

Although the net charge on the derivatized Lys residue was maintained, peptide mapping analysis showed that amidination of ε-amino groups of lysine prior to digestion, precluded trypsin proteolysis at Lys-C position. The procedure used proved to be useful for decreasing the complexity of the peptide map, favoring quantitative evaluation.

1.2.4. Quantification by MALDI-TOF Mass Analysis

The observed and expected ratios are very similar. Quantification of relative change in protein concentration can be achieved by such a differential labeling.

See FIG. 3.

Example 1 shows that the total amidination of lysines enables quantitative comparisons of proteins mixture. It also shows that labeled proteins could be separed by 2D electrophoresis.

### Example 2

### Identification of Labelled Proteins in an Extract of Arabidopsis thaliana

2.1 Materials and Methods

Experiments were performed on soluble proteins of cell suppressors of Arabidopsis thaliana cultured in normal conditions.

Samples have been solubilized for derivatization: 50 μg of the arabidopsis extract is dissolved in 20 μl of 6M urea, 4% 3-[(3-Cholamidopropyl)dimethylammonio]-1-propanesulfonate (CHAPS). Protein was incubated at room temperature during 1 h 30 in the presence of 10 μl of either S-Methyl thioacetimidate and S-Methyl thiopropionomidate reagent (1 mg in 50 μl of 150 mM NaHCO₃). 3 μl of ice cold TCA (trichloro acetic acid) were added to the mixture and left 2 h. at 4° C. 38 000 g/30 min. centrifugation was then performed. Trichloroacetic acid precipitates were then washed with three times with diethyl ether.

The extract was then analysed by 2D electrophoresis. A spot of proteins selected because of the diversity of the molecular mass and isoelectric spot, was excised for analysis with MALDI-TOF/TOF MS/MS. The proteins were identified on the basis of their peptide mass fingerprint by using Mascot (a search engine), and Arg-C peptidase as enzyme.

2.2. Results

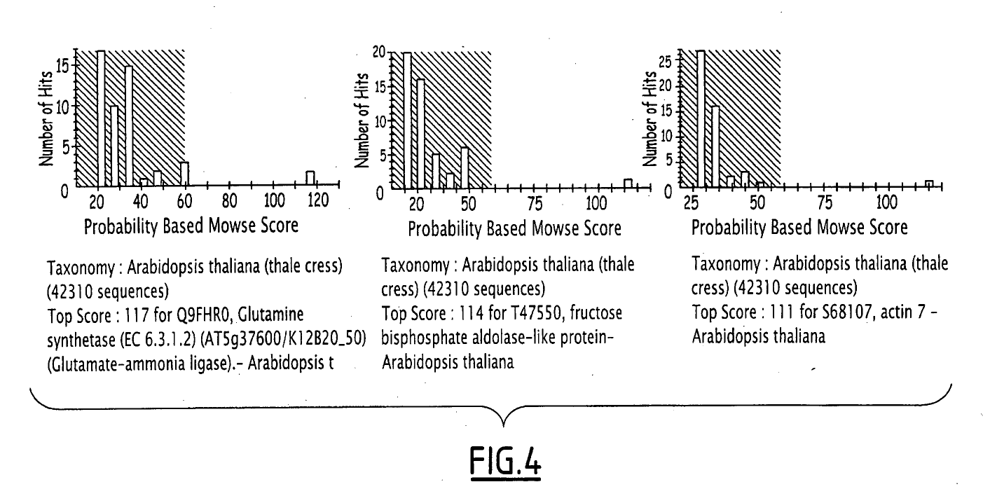

The results of the identification of the proteins are shown in FIG. 4. There is a good correlation with the reference note.

What is claimed is:

1. A method for comparative analysis of two or more samples to determine ratios of the amounts of proteins of the samples, which method comprises the steps of:
(i)
labeling ε-amino groups of lysine residues of the proteins of a first sample, with a first amidination agent;
labeling ε-amino groups of lysine residues of the proteins of a second sample, with a second amidination agent;
wherein the first amidination agent and the second amidination agent are selected to give a desired mass shift by mass spectrometry;
(ii) mixing pools of the first and second samples;
(iii) separating the labeled proteins;
(iv) cleaving the separated proteins;
(v) subjecting the cleaved proteins to mass spectrometry analysis.

2. The method of claim 1, wherein the separating step (iii) is performed by 2D gel electrophoresis.

3. The method of claim 1, wherein the first amidination agent is a S-alkyl thioacylimidate, wherein the acyl group is selected from the group consisting of acetyl, propionyl, butyryl, valeryl, hexynyl, isobutyryl and isovaleryl, and wherein the alkyl group comprises from 1 to 4 carbon atoms.

4. The method of claim 3, wherein the thioalkylimidate is S-methyl thioacetamidate.

5. The method of claim 3, wherein the thioalkylimidate is S-methyl thiopropionimidate.

6. The method of claim 1, wherein the first amidination agent differs from the second amidination agent by at least a methylene group.

7. The method of claim 6, wherein the first amidination agent is S-methyl thioacetamidate and the second amidination agent is S-methyl thiopropinimidate.

8. The method of claim 1, wherein the cleavage step iv) is a trypsin digestion.

9. The method of claim 1, wherein the mass spectrometry is a MALDI-TOF analysis.

10. The method of claim 1, wherein the samples are plant extracts.
